(19) United States
(12) Patent Application Publication
(10) Pub. No.: US 2026/0221252 A1
(43) Pub. Date: Jul. 30, 2026

(54) A PERSONAL CARING AND LEARNING SYSTEM AND METHOD

(71) Applicant: ALICE WELLBEING OY, Helsinki (FI)

(72) Inventors: Aleksi LAAKKONEN, Espoo (FI); Göte NYMAN, Helsinki (FI); Antti KIRJALAINEN, Helsinki (FI); Christian BERGSTRÖM, Tammisaari (FI); Krista LAGUS, Espoo (FI)

(21) Appl. No.: 19/157,891

(22) PCT Filed: Feb. 21, 2023
(86) PCT No.: PCT/FI2023/050103

§ 371 Date: Aug. 19, 2025

(51) Int. Cl. G16H 20/00; G06F 21/62; G16H 10/20; G16H 10/60

(52) U.S. Cl. CPC G16H 20/00; G06F 21/6245; G16H 10/20; G16H 10/60

(57) ABSTRACT

An object of the invention is a personal caring and learning system configured to artificial intelligence based management of personal wellbeing and health comprising: an electronic device (100) including a processor (102) and an artificial intelligence system (104); measurement units (106) for measuring data of physical and mental characteristics of a person, and data of situations, behavior and experiences of a person. The system comprises means (116) of the artificial intelligence system (104) for receiving and analyzing data from the measurement units (106) and from the external data asset connecting protocols (108), products (110) and services (114); a confidential communication robot (118) of the artificial intelligence system (104) for personal, AI-confirmatory and confidential communication between the person and the robot to form communication data; an integration unit (120) of the artificial intelligence system to integrate data from the means (116) of the artificial intelligence system (104) and from the confidential communication robot (118) to process value data for mental and physical health benefits defined for a person profile and situations; storing means (122) for storing data to form personal mental and physical data history; and a learning unit (124) of the artificial intelligence system (104) for system learning from the mental and physical data history.

### FIELD OF THE INVENTION

The present invention relates to the field of systems and methods that support the management of personal wellbeing and health and evokes personal interest in them.

### BACKGROUND OF THE INVENTION

Health issues are on the rise. One out of eight people in our planet live with a mental health condition—the total number of people living with a mental disorder is nearly I billion people. Also, overweight and obesity are major risk factors for chronic diseases such as cardiovascular disease, diabetes, musculoskeletal disorders and some cancers.

Health problems are associated with large direct costs for individuals and society, such as the provision of health and social care.

On average in the EU, public and private expenditure on preventive care accounted for 2.8% of total health expenditure in 2018. In other words, 97% of healthcare expenditure is spent on fixing injuries and diseases and only about 3% is being spent on preventive care that aims to avoid or reduce the number or the severity of injuries and diseases, their consequences and complications. Also, economic overload due to this problem is already too much for most of the countries.

The path to improved global health requires more than an increase in the use of medication. Rather, an effective solution likely lies in prevention; motivating, supporting, and equipping individuals to make healthy lifestyle choices.

Making informed decisions can be a challenging exercise, especially with the amount of complexity that is often involved. This invention seeks to reduce this difficulty by providing tools to empower individuals in making better informed choices confidently.

Patent document U.S. Pat. No. 10,991,463 B2 presents computer-implemented system and methods for predicting the health and therapeutic behavior of individuals using artificial intelligence, smart contracts and blockchain.

None of the prior art discloses a system which provides a preventive and confidential personal wellbeing relationship to prevent problems in health and wellbeing with the use of physical data, mental data, and behavior data.

### SUMMARY OF THE INVENTION

It is an object of the invention to ensure personal wellbeing, to promote satisfying and healthy life habits and to provide proactive prevention of problems in these. This is achieved by a personal learning system for making artificial intelligence based preventive and caring lifestyle recommendations comprising an electronic device including a processor and an artificial intelligence system and measurement units for measuring data of physical and mental characteristics of a person. The system is connected to the measurement units and to the external data asset connecting protocols, products and services. The system comprises means of the artificial intelligence system for receiving and analyzing data from the measurement units and the external data asset connecting protocols, products and services, confidential communication robot of the artificial intelligence system for personal and confidential communication between the person and the robot to form communication data, integration unit of the artificial intelligence system to integrate data from the means of the artificial intelligence system and from the confidential communication robot to process value data for mental and physical health benefits defined for a person profile and situations, storing means for storing data to form personal data history and learning unit of the artificial intelligence system for system learning from the personal data history.

An object of the invention is also a personal caring and learning method of making artificial intelligence based preventive and caring lifestyle recommendations comprising: measuring data of physical and mental characteristics of a person, and data of situations, behavior and experiences of a person. The method comprises using of connections to measurement units and to external data asset connecting protocols, products and services; performing AI based analysis to data from the measurement units and from the external data asset connecting protocols, products and services; conducting of personal, AI-confirmatory and confidential communication between the person and a confidential communication robot to form communication data; integrating data from the artificial intelligence system and from the confidential communication robot to process value data for mental and physical health benefits defined for a person profile and situations; storing data to form personal mental and physical data history; and performing system learning from the mental and physical data history.

The invention is based on data from measurement units and to external data asset connecting protocols, products, services and means of the artificial intelligence system for receiving and analyzing said data. The invention is also based on confidential communication robot of the artificial intelligence system for personal and confidential communication between the person and the robot to form communication data. The invention is further based on integration unit of the artificial intelligence system to integrate data from the means of the artificial intelligence system and from the confidential communication robot to process value data for the person and storing means for storing data to form personal data history and learning unit of the artificial intelligence system for system learning from the personal data history.

The invention can also be based on confidential communication robot of the artificial intelligence system for personal and confidential communication between the person and the robot to form communication data. Confirmatory AI can be used in the person-robot interaction to ensure that the system and the robot have correct and relevant data about the person.

Benefits of the invention are personal healthy lifestyle and wellbeing, automated preventive health system for a society and huge cost savings for countries and individuals.

### BRIEF DESCRIPTION OF THE DRAWINGS

FIG. 1 presents an exemplary wellbeing system according to the present invention.

FIG. 2 presents exemplary method phases according to the present invention.

FIG. 3 presents exemplary system architecture and connections between the blocks according to the present invention.

### DETAILED DESCRIPTION OF THE INVENTION

Contextualized and confirmatory learning AI engine can perform e.g. the following method tasks: 1. Analyzes customer inputs. 2. Compares them to existing peer models (obtained by pattern matching from Peer modeling AI engine), and locates potential peer models. 3. Asks questions and obtains reinforcement signal from confirmatory conversation strategies and user-chosen protocols, to identify the context-appropriate peer model(s), and then, the context-appropriate intervention protocols to propose. 4. By utilizing the length and success of disambiguating conversation with user as an optimization criterion, learns to adapt confirmatory conversation strategies towards more quickly proposing user-appropriate and user-satisfying protocols.

Peer modeling AI engine can learn a set of generalized customer models based on previously stored customer data set. The content data it works on may include mental, physical, situational and physical environment data, as well as conversations, user-accepted protocol history, and user feedback from experienced protocols so far. The modeling phase utilizes data records without personal identification data—instead, customer id: s are pseudonyms at this stage. The already learned model is able to return the closest-matching abstracted peer models for a given situated customer data record. Based on Peer models is given predictions of future outcomes of potential new contextual episodes, life events, intervention protocols or services

The present invention is generally directed to individualized wellbeing and management of health and wellbeing. In the embodiments according to the present invention is combined physiological measurements with interactive communication with AI (artificial intelligence) robot to understand person's current physical state, mental state and preferred states to be achieved. The system can be configured to analyze and understand person's status and objectives to provide with e.g. a custom set of protocols, products and services which have the highest probability to improve wellbeing.

The system according to the present invention is designed to inspire, guide and motivate the person using it. The system aims at promoting personal behavior, self-control, experiences, and thinking so that the person will be advanced in self-perception, personal well-being, social relationships and health. The system can collect informative qualitative and quantitative behavior and experience data about the person and in return the system can provide trustworthy information and guidance to the person based on relevant professional knowledge, peer model information and interpretation.

The system according to the present invention can collect personal physiological tracker data and combine the tracker data with data of subjective experiences, comments, interpretations and reactions in the AI robot discussions. The system can use AI to mine IoT (Internet of Things) data and IoB (Internet of Behaviors) data and utilizes relevant psychological framework data to determine the best evidence-based wellbeing guidance to the person. Adaptive e-learning methods and virtual assistants can be used to improve person's learning and wellbeing outcomes. The system can be configured to communicate with the person in a personal, inspiring and acceptable way, and also achieving legal and personal privacy needs. The system can be configured to convergent interaction with the person who then confirms that the system has understood the person correctly or if needed, she or he can correct some detail or details or interpretation with the system. The person can find an individual path to wellbeing with the system. Preferably, key measurement metrics are subjective happiness, life satisfaction, emotional balance and heart health.

In the preferred embodiments according to the present invention, the system can be configured to be modular, dynamic and interconnected. The system can utilise proprietary technology interconnected with external technology modules and data sources to be able to harness the power of the most advanced digital assets available. The data to be analyzed is formed in integrated processes by listening the person to form informative data and analyzing her or his personal senses to form sensitive data and by collecting data from measurement units and from external data sources, like weather and location services, health data producers, personal trainer database, doctor database, psychology database, etc, to detect, process and analyze correlations with external and internal databases.

The system according to the present invention comprises artificial intelligence (AI) communication robot for personal and confidential communication between the person and the robot to form communication data. The AI robot can comprise microphone means to receive person's speech data and preferably also other noise data, means for receiving sensory and gestural data, and AI algorithms to process and analyze the received data. Other noise data can be e.g. external sounds such as dog, baby, renovation, sea, etc, noise levels, relation of different sounds to other noise components and relation to other data components in the system.

The AI robot can also comprise eye means for receiving visual data. The visual data is processed and analyzed in AI algorithms: e.g. recognition of age, gender, emotions, eye focus, etc, external objects and environment such as dog, baby, renovation, sea, etc, relation to other visual data components and relation to other data components in the system. The AI robot can further comprise AI speaking means for fixed dynamic speech responses to the person. The AI based speaking means can comprise e.g. a speech synthesizer to form speech on basis of AI analyzed information and a confirmatory language understanding system. In communication with the person the AI robot (agent) can collect personal and confidential data on psychological measurements such as e.g. PERMA results, subjective states and experiences, situations, eating habits, diet, allergies, worries, goals, dreams and visions.

The system according to the present invention comprises measurement means to collect personal data such as data e.g. on movements, subjective experiences and interpretations, sleep characteristics, HRV (heart rate variability), blood, sugar, oxygen levels, etc. Also, personal data such as cognitive test results, work history, personal situations, hobbies, etc. can be collected. The collected personal data is analyzed and compared to other data components in AI algorithms.

The system according to the present invention comprises also means to collect external data such as data on location (city, countryside), weather galactic movements, application information (social media, news, meditation, training, etc.), medical conditions, drugs, vaccinations, etc. Said external data is analyzed and compared to other data components in AI algorithms.

Embodiments according to the present invention enable that personal wellbeing knowledge leads to motivation for creating positive habits. Customized and practical wellbeing protocols support wellbeing. Easy and automated measurements guide self-development progress. The embodiments also enable that the person can own personal wellbeing data and sell it anonymously for financial gains.

Embodiments according to the present invention also enable that the subject can be informed about data assets connecting protocols, products and services with measurable and known mental and physical health benefits for defined user profiles and situations. The subject can then choose a wellbeing protocol, product and service by contacting these providers. This can be applied so that a safe automatic promotion and sales of protocols are possible. Products and services can also be provided user-specifically to users who, based on scientific knowledge and/or peer model information, are most likely to gain higher health benefits.

For society savings on health and social care costs can be huge because of more balanced, productive citizens and communities. Also, the system allows the offering of anonymized wellbeing data for research, education and any beneficial public allocating purposes.

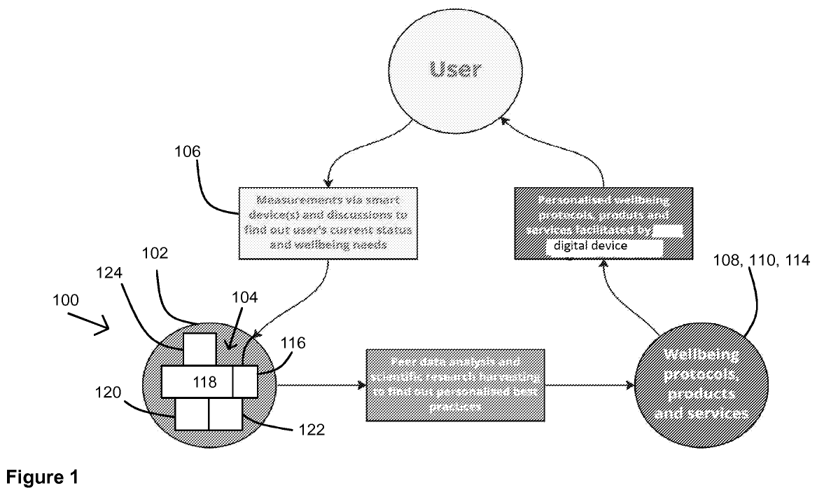

In the following is referred to the exemplary FIGS. 1-3 and described exemplary embodiments according to the present invention. FIG. 1 presents a personal learning system for making artificial intelligence based preventive and caring lifestyle recommendations. The system comprises an electronic device 100 including a processor 102 and an artificial intelligence system 104. The system also comprises measurement units 106 for measuring data of physical and mental characteristics of a person. The system is connected to the measurement units 106 and to external data asset connecting protocols 108, products 110 and services 114. In a preferred embodiment, a structure of the system is modular and interconnected comprising external technology modules and data sources.

The system further comprises means 116 of the artificial intelligence system 104 for receiving and analyzing data from the measurement units 106 and from external data asset connecting protocols 108, products 110 and services 114. A confidential communication robot 118 of the artificial intelligence system 104 is configurated for personal and confidential communication between the person and the robot to form communication data. The confidential communication robot 118 can be configured to verify data received from the person by asking verification questions from the person. The system comprises preferably means for securing personal confidentiality and privacy needs. The means can be accomplished e.g. by a Peer modeling engine which is not being re-learned, and thus not storing any customer's data directly. Rather, it stores a generalized, abstracted, condensed version of the original full customer data set. This provides a layer of data privacy as it protects individual customer data by abstracting away from it. Also, the means for securing can be accomplished e.g. by https related securing methods and/or by PKS (public key securing) methods.

In one exemplary embodiment the system can comprise a health database, which the person willingly provides, including data of the person, wherein the health data of the person includes one or more existing health conditions, one or more limiting health factors, and a medical compliance record for the existing health condition, and one or more health conditions first time present in a therapy regime of the person.

In a preferred embodiment the system comprises the means 116 of the artificial intelligence system 104 for analyzing collected informative qualitative and quantitative behavior and experience data from the person to provide qualified and classified information and guidance to the person based on relevant professional knowledge, peer model information and interpretation.

In FIG. 1 is also presented an integration unit 120 of the artificial intelligence system according to the present invention to integrate data from the means 116 of the artificial intelligence system 104 and from the confidential communication robot 118 to process value data for mental and physical health benefits defined for a person profile and/or for situations. In a preferred embodiment the integration unit 120 combines physiological measurements data from the means 116 of the artificial intelligence system 104 with interactive communication data from the confidential communication robot 118 of the artificial intelligence system 104 to analyze current physical state, mental state and preferred states of the person to be achieved to provide the person with a custom set of protocols, products and services which have the highest probability to improve wellbeing.

The system according to the present invention also comprises storing means 122 for storing data to form personal mental and physical data history and a learning unit 124 of the artificial intelligence system 104 for system learning from the mental and physical data history. Personal data is collected by the confidential communication robot 118 and it can be systematically stored as personal life and experience data so that it can be used for statistical and machine learning analysis. This makes possible the storage, analysis, learning and other beneficial uses of significant life event data. In one preferred embodiment the learning unit 124 of the artificial intelligence system 104 is configured for combining personal mental and physical data history with the communication data about subjective comments and reactions for analysis in an adaptive e-learning process to improve person's learning and wellbeing outcomes.

Preferably, the system can be configured to integrate AI (Artificial Intelligence) data, IoT (Internet of Things) data, IoB (Internet of Behaviors) data and personal mental and physical data history to determine the best evidence-based wellbeing guidance to the person. In one preferred embodiment the system can be configured to use cryptocurrency technologies or other technologies in rewarding the person to ensure preventive wellbeing objectives. A master digitalization platform can be configured to digitalize wellbeing specialists and their knowledge to be shared via the system to the users. Wellbeing entrepreneurs (life coaches, mindfulness teachers, therapists etc.) have a possibility to digitalize themselves, their wellbeing knowledge and protocols. Effects of such protocols can be measured with voluntary test subjects recruited via the system. If measurements show statistically relevant positive impact on subjects'wellbeing, teachings and protocols are validated to be shared or sold via the system. Also, the users (persons) have a possibility to digitalize themselves and personal wellbeing knowledge so that they can share their wellbeing insights and teachings.

In the embodiments according to the present invention can be utilized compensation models to ensure that system results will be achieved more effectively. In an exemplary user incentivation model users can be compensated for most activity within system with in-system virtual currency. Activities can include but are not limited to the following: time spent using system, answered questions, tested protocols, stored data, marketing of system, community engagement. Users can be compensated for choosing to share their data for research purposes, for government agencies and/or for companies in fields such as insurance, pharma, wellbeing and wellness products and services. In-system virtual currency can be used to make purchases within the system as well as converted to other supported virtual currency (for example Bitcoin or Ethereum) or to fiat currency.

In an exemplary creator incentivation model protocol creators can be compensated with in-system virtual currency for sale of their protocols. In-system virtual currency can be converted to other supported virtual currency (for example Bitcoin or Ethereum) or to fiat currency. A creator is defined in this context as any user who is not a merchant partner. Merchant partners follow a distinct model.

In an exemplary affiliate and merchant model affiliate sales and sales of merchant partner protocols can be compensated. The affiliate program allows people to earn money by promoting the system and protocols (activities, products and services). Affiliates can use their own website, blog, social media pages, or other online platforms to promote protocols and generate sales. When someone makes a purchase, the affiliate earns a commission. The merchant model allows partners to create their own presence within the system, and list protocols within the system marketplace. Merchants pay a fee for each item sold, as well as for access to other premium features like promotional tools. This model gives entrepreneurs control of their own businesses with the benefit of system's reach, targeting and customer base.

In an exemplary smart contract model, a smart contract is a computer program or a transaction protocol that is intended to automatically execute, control or document events and actions according to the terms of a contract or an agreement. In the system smart contract are digitally signed between the system, users, creators, affiliates and merchants. System can be using any notable blockchain platform, for example Ethereum, Bitcoin or Cardano.

Smart contact embodiments can include but are not limited to the following:
1. User selling personal data to a research party, governmental agency or a company: Buying party can make an offer via the system to relevant users and set the buying price freely. If the offer is accepted by the user, system automatically creates a smart contract between the system, creator and the user regarding transfer of data and currency. In one exemplary embodiment the system can transfer the currency and the data when the smart contract is accepted. System can take a transaction fee for facilitating the data and currency transfers if the smart contract between the parties so states.
2. Creators selling protocols: Creators can set the price of a protocol freely and an offer it via the system to relevant users. When a protocol is bought by the user, system automatically creates a smart contract between the system, creator and the user regarding transfer of protocol, data and currency. System transfers the protocol and the currency when the smart contract is accepted. When the user is executing the protocol, system can transfer data to the creator for validation and development purposes of the protocol. System can take a transaction fee for facilitating the data and currency transfers if smart contract between the parties so states.
3. Affiliates promoting the system, protocols, products and services: System and the owners of wellbeing protocols, products or services can freely make offers for affiliates regarding sales commission. If the offer is accepted by the affiliate, system automatically creates a smart contract between the system, affiliate and the owner regarding transfer of protocols, products, services and currency. System transfers the currency when the smart contract conditions are met. System can take a transaction fee for facilitating protocol, product, service and currency transfers if the smart contract between the parties so states.
4. Merchant provides and promotes an inventory of protocols, products and services in the system: Merchants can make their wellbeing inventory available in the system. When an inventory is included in the system, a smart contract is created between a merchant and the system regarding a transaction fee for facilitating protocol, product, service, currency transfers and promotional services.

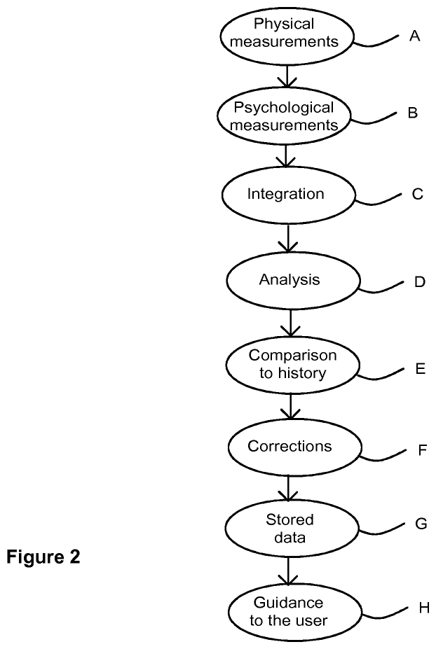

In FIG. 2 is presented an exemplary method flow chart according to the present invention. In the exemplary method of the personal learning system presented in FIG. 1: A) is measured data of physical and mental characteristics (e.g. HRV, pulse, blood pressure, blood sugar, body temperature, noise measurements, eye measurements, etc.) to form personal health data, B) is formed personal mental state data in confidential communication with the robot (118 in FIG. 1), C) is integrated the data formed in steps A&B to form data to artificial intelligence analysis by utilizing information provided by outside providers connected to the system, D) is analyzed the integrated data formed in step C to form value data for mental and physical health benefits defined for a person profile and situations, E) is compared the formed value data to personal mental and physical data history, F) is made corrections to the value data, G) the corrected value data is stored in storing means (122 in FIG. 1), and H) on basis of the corrected value data is determined and given the best evidence-based wellbeing guidance to the person. E.g. method steps A and B can be in different order. Also, e.g. method steps G and H can be in different order.

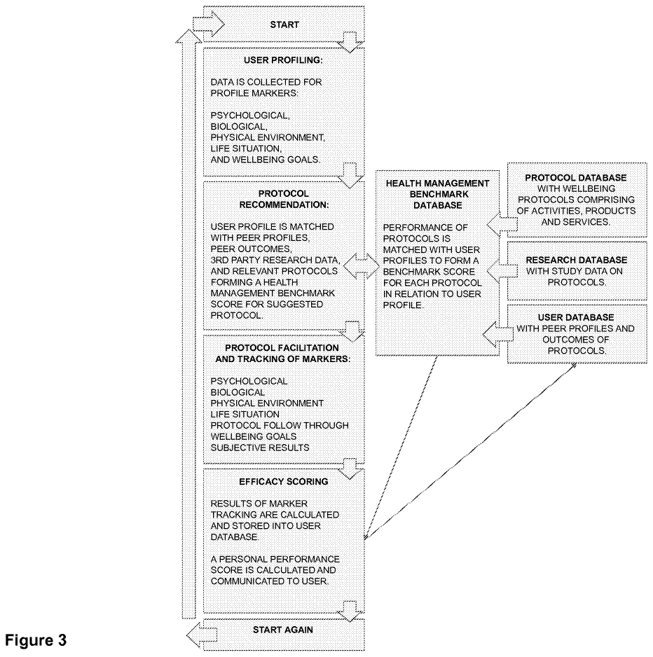

FIG. 3 presents exemplary system architecture and connections between the blocks according to the present invention. Anyway, the invention can be implemented with different kind of variations in system architectures and in connections between the blocks, so embodiments according to the invention are not limited to the exemplary FIG. 3.

The presented artificial intelligence based means, e.g. 104, 118, 120, 122 and 124 for performing different kind of tasks according to the present invention can be carried out programmatically by utilizing e.g. algorithm techniques by means of data processor techniques. The presented artificial intelligence based means, e.g. 104, 118, 120, 122 and 124 can be located e.g. in a mobile device (phone, tablet, etc.) or in a personal computer or in some other digital device.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

It is also contemplated that the system presented in FIG. 1 is operable to not include all of the components shown in FIG. 1, is operable to include other components that are not explicitly shown in FIG. 1, or is operable to utilize an architecture completely different than that shown in FIG. 1. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons in the art may implement the described functionality in varying ways for each particular application (e.g., arranged in a different order or partitioned in a different way), but such implementation decisions should not be interpreted as causing a departure from the scope of the present invention.

The above-mentioned examples are provided to serve the purpose of clarifying the aspects of the invention, and it will be apparent to one skilled in the art that they do not serve to limit the scope of the invention. By nature, this invention is highly adjustable, customizable and adaptable. The above-mentioned examples are just some of the many configurations that the mentioned components are able to take on. All modifications and improvements have been deleted herein for the sake of conciseness and readability but are properly within the scope of the present invention.

1. A personal caring and learning system configured to artificial intelligence based management of personal wellbeing and health comprising: an electronic device (100) including a processor (102) and an artificial intelligence system (104); measurement units (106) for measuring data of physical and mental characteristics of a person, and data of situations, behavior and experiences of a person, characterized in that the system being connected to the measurement units (106) and to external data asset connecting protocols (108), products (110) and services (114); the system comprising: means (116) of the artificial intelligence system (104) for receiving and analyzing data from the measurement units (106) and from the external data asset connecting protocols (108), products (110) and services (114); a confidential communication robot (118) of the artificial intelligence system (104) for personal, AI-confirmatory and confidential communication between the person and the robot to form communication data; an integration unit (120) of the artificial intelligence system to integrate data from the means (116) of the artificial intelligence system (104) and from the confidential communication robot (118) to process value data for mental and physical health benefits defined for a person profile and situations; storing means (122) for storing data to form personal mental and physical data history; and a learning unit (124) of the artificial intelligence system (104) for system learning from the mental and physical data history.

2. The personal learning system according to the claim 1, characterized in that the system comprises the integration unit (120) for combining physiological measurements data from the means (116) of the artificial intelligence system (104) with interactive communication data from the confidential communication robot (118) of the artificial intelligence system (104) to analyze current physical state, mental state and preferred states of the person to be achieved to provide the person with a custom set of protocols, products and services which have the highest probability to improve wellbeing.

3. The personal learning system according to the claim 1, characterized in that the system comprises a health database, which the person willingly provides, including data of the person, wherein the health data of the person includes one or more existing health conditions, one or more limiting health factors, and a medical compliance record for the existing health condition, and one or more health conditions first time present in a therapy regime of the person.

4. The personal learning system according to the claim 1, characterized in that the system is modular and interconnected comprising external technology modules and data sources.

5. The personal learning system according to the claim 1, characterized in that the system comprises the means (116) of the artificial intelligence system (104) for analyzing collected informative qualitative and quantitative behavior and experience data from the person to provide qualified and classified information and guidance to the person based on interpretation and at least one of relevant professional knowledge and peer model information.

6. The personal learning system according to the claim 1, characterized in that the learning unit (124) of the artificial intelligence system (104) is configured for combining personal mental and physical data history with the communication data about subjective comments and reactions for analysis in an adaptive e-learning process to improve person's learning and well-being outcomes.

7. The personal learning system according to the claim 1, characterized in that the system is configured to integrate AI (Artificial Intelligence) data, IoT (Internet of Things) data, IoB (Internet of Behaviors) data and personal mental and physical data history to determine the best evidence-based wellbeing guidance to the person.

8. The personal learning system according to the claim 1, characterized in that the system comprises means for securing personal confidentiality and privacy needs.

9. The personal learning system according to the claim 1, characterized in that the confidential communication robot (118) is configured to verify data received from the person by asking verification questions from the person.

10. The personal learning system according to the claim 1, characterized in that the system is configured to use cryptocurrency technologies in rewarding the person to ensure preventive wellbeing objectives.

11. A personal caring and learning method of making artificial intelligence based preventive and caring lifestyle recommendations comprising: measuring data of physical and mental characteristics of a person, and data of situations, behavior and experiences of a person; characterized in that the method comprises: using of connections to measurement units (106) and to external data asset connecting protocols (108), products (110) and services (114); performing AI based analysis to data from the measurement units (106) and from the external data asset connecting protocols (108), products (110) and services (114); conducting of personal, AI-confirmatory and confidential communication between the person and a confidential communication robot (118) to form communication data; integrating data from the artificial intelligence system (104) and from the confidential communication robot (118) to process value data for mental and physical health benefits defined for a person profile and situations; storing data to form personal mental and physical data history; and performing system learning from the mental and physical data history.

12. The personal caring and learning method according to the claim 11, characterized in that the method is combined physiological measurements data from the artificial intelligence system (104) with interactive communication data from the confidential communication robot (118) of the artificial intelligence system (104) to analyze current physical state, mental state and preferred states of the person to be achieved to provide the person with a custom set of protocols, products and services which have the highest probability to improve wellbeing.

13. The personal caring and learning method according to the claim 11, characterized in that in the method is provided a health database, which includes data of the person, wherein the health data of the person includes one or more existing health conditions, one or more limiting health factors, and a medical compliance record for the existing health condition, and one or more health conditions first time present in a therapy regime of the person.

14. The personal caring and learning method according to the claim 11, characterized in that the method is performed modularity and interconnectivity comprising external technology modules and data sources.

15. The personal caring and learning method according to the claim 11, characterized in that the system comprises the means (116) of the artificial intelligence system (104) for analyzing collected informative qualitative and quantitative behavior and experience data from the person to provide qualified and classified information and guidance to the person based on based on interpretation and at least one of relevant professional knowledge and peer model information.

16. The personal caring and learning method according to the claim 11, characterized in that in the method is combined personal mental and physical data history with the communication data about subjective comments and reactions for analysis in an adaptive e-learning process to improve person's learning and well-being outcomes.

17. The personal caring and learning method according to the claim 11, characterized in that in the method is integrated AI (Artificial Intelligence) data, IoT (Internet of Things) data, IoB (Internet of Behaviors) data and personal mental and physical data history to determine the best evidence-based wellbeing guidance to the person.

18. The personal caring and learning method according to the claim 11, characterized in that in the method is secured personal confidentiality and privacy needs.

19. The personal caring and learning method according to the claim 11, characterized in that in the method is configured the confidential communication robot (118) to verify data received from the person by asking verification questions from the person.

20. The personal caring and learning method according to the claim 11, characterized in that in the method is used cryptocurrency technologies in rewarding the person to ensure preventive wellbeing objectives.
